(19) United States
(12) Patent Application Publication
(10) Pub. No.: US 2026/0114801 A1
(43) Pub. Date: Apr. 30, 2026

(54) MEDICAL EQUIPMENT, DETECTION METHOD, AND NON-TRANSITORY STORAGE MEDIUM STORING PROGRAM THEREOF

(71) Applicant: FUKUDA DENSHI CO., LTD., Tokyo (JP)

(72) Inventor: Mai KOBAYASHI, Tokyo (JP)

(73) Assignee: FUKUDA DENSHI CO., LTD., Tokyo (JP)

(21) Appl. No.: 19/364,787

(22) Filed: Oct. 21, 2025

(30) Foreign Application Priority Data
Oct. 31, 2024 (JP) 2024-191663

(51) Int. Cl. A61B 5/00; A61B 5/087; G16H 40/67

(52) U.S. Cl. CPC A61B 5/4818; A61B 5/087; A61B 5/7282; G16H 40/67

(57) ABSTRACT

Medical equipment for detecting abnormal respiration of a patient during sleep includes an identification unit that identifies respiratory amplitudes of a plurality of times of respiration conducted by the patient in a respiratory period from elimination of apnea or hypopnea to a next occurrence of the apnea or the hypopnea, a calculation unit that calculates an approximation function that approximates the respiratory amplitudes of the plurality of times of the respiration, and a determination unit that determines that the abnormal respiration occurs in the patient, in a case in which a predetermined condition relating to the approximation function is satisfied.

### BACKGROUND

The present disclosure relates to medical equipment, a detection method, and a non-transitory storage medium storing program thereof.

The Cheyne-Stokes respiration (CSR) which is central abnormal respiration in which hyperpnea and hypopnea or apnea are periodically repeated may occur in some of heart failure patients who have sleep disordered breathing. In Japanese Patent No. 5478017, there is described a technology of analyzing a frequency spectrum of respiratory data, thereby detecting the CSR.

### SUMMARY

Analysis of the respiratory data in the frequency domain is lower in calculation efficiency than analysis of the respiratory data in the time domain. As a result, an amount of use of a memory and an amount of calculation of a processor are large. One aspect of the present disclosure has an object to provide a technology for efficiently detecting abnormal respiration of a patient during sleep.

In some embodiments, there is provided medical equipment for detecting abnormal respiration of a patient during sleep. The medical equipment includes an identifier configured to identify respiratory amplitudes of a plurality of times of respiration conducted by the patient in a respiratory period from elimination of apnea or hypopnea to a next occurrence of the apnea or the hypopnea, a calculator configured to calculate an approximation function that approximates the respiratory amplitudes of the plurality of times of the respiration, and a determiner configured to determine that the abnormal respiration has occurred in the patient in a case in which a predetermined condition relating to the approximation function is satisfied.

According to some embodiments, it is possible to efficiently detect the abnormal respiration of the patient during sleep.

### BRIEF DESCRIPTION OF THE DRAWINGS

FIG. 1 is a block diagram for illustrating a configuration example of hardware of some embodiments;

FIG. 2A is a schematic diagram for illustrating characteristics of respiration of a patient contracting central sleep apnea (CSA);

FIG. 2B is a schematic diagram for illustrating characteristics of respiration of a patient contracting obstructive sleep apnea (OSA);

FIG. 3 is a flowchart for illustrating a detection method example according to some embodiments;

FIG. 4A is a schematic diagram for illustrating a detection method example according to some embodiments;

FIG. 4B is a schematic diagram for illustrating a detection method example according to some embodiments;

FIG. 5A is a schematic diagram for illustrating a modification example of the detection method according to some embodiments; and

FIG. 5B is a schematic diagram for illustrating a modification example of the detection method according to some embodiments.

### DETAILED DESCRIPTION OF THE PREFERRED EMBODIMENTS

A detailed description is now given of embodiments with reference to the drawings. Note that the following embodiments do not limit the disclosure according to claims and all of combinations of features described in the embodiments are not necessarily essential for the disclosure. Two or more features among the plurality of features described in the embodiments may freely be combined with one another. Moreover, the same or similar configurations are denoted by the same reference signs, and a redundant description thereof is omitted.

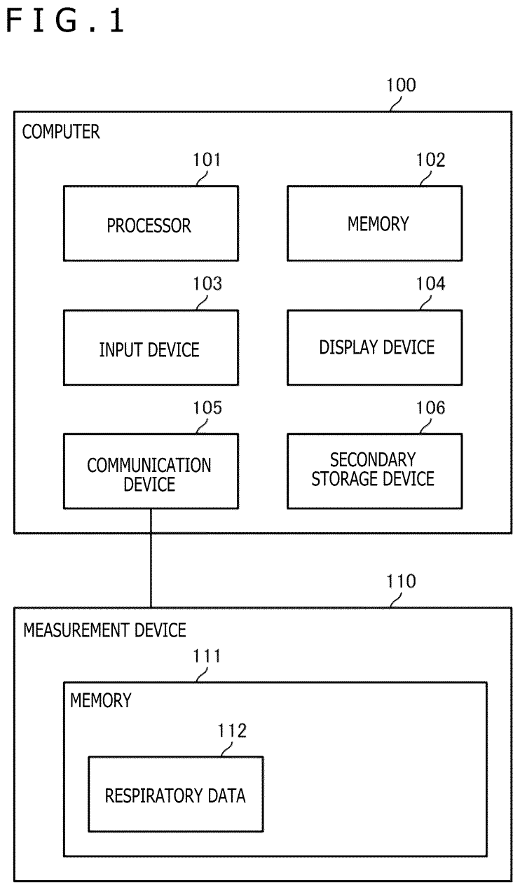

With reference to FIG. 1, a description is given of a hardware configuration example of a computer 100 according to some embodiments. As described in detail hereinafter, the computer 100 is used to detect abnormal respiration of a patient during sleep. Thus, the computer 100 may be referred to as medical equipment, a detection device, an abnormal respiration detection device, or the like. In a description given below, as an example of the abnormal respiration of the patient during sleep, the CSR is dealt with. Alternatively, the computer 100 may be used to detect other types of abnormal respiration. The computer 100 may estimate that the patient has disease accompanied by the CSR, on the basis of detection of the CSR. The computer 100 may be, for example, a server computer or a personal computer (of, for example, a desk-top or lap-top type). The computer 100 may be a computer resource disposed in a cloud environment. The computer 100 may be a dedicated computer for executing medical processing including the detection of the CSR.

The computer 100 may include a hardware device illustrated in FIG. 1. A processor 101 controls an overall operation of the computer 100. The processor 101 may be formed of, for example, a central processing unit (CPU), a graphic processing unit (GPU), or a combination thereof. The processor 101 may be a single processor or a set of a plurality of processors connected with one another for mutual communication.

A memory 102 stores programs and data used for processing of the computer 100. The memory 102 may be formed of, for example, a combination of a random access memory (RAM) and a read-only memory (ROM).

An input device 103 is a device for acquiring an instruction from a user of the computer 100. The input device 103 may be formed of, for example, a combination of one or more of a keyboard, a button, a touchpad, and a microphone. A display device 104 is a device for visually presenting information to the user of the computer 100. The display device 104 may be a display of the dot-matrix type, such as a liquid crystal display, for example. The computer 100 may include a device (for example, a touch screen) integrally formed of the input device 103 and the display device 104. The input device 103 and the display device 104 may exist outside the computer. In this case, the computer 100 may include an interface for communicating with the external input device 103 and the display device 104.

A communication device 105 is a device for communicating with a device outside the computer 100. In a case in which the computer 100 executes wired communication, the communication device 105 may be a network interface card (NIC) having a connector for connecting a cable. In a case in which the computer 100 executes wireless communication, the communication device 105 may be a wireless communication module including an antenna and a baseband processing circuit.

A secondary storage device 106 is a device which stores, in a nonvolatile manner, programs and data used for the processing of the computer 100. The secondary storage device 106 is formed of, for example, a hard disk drive (HDD) or a solid state drive (SSD).

The computer 100 may be communicable with a measurement device 110. The measurement device 110 is a device capable of measuring a respiratory waveform of the patient during sleep. The respiratory waveform measured by the measurement device 110 may be a waveform indicating an airflow of the respiration of the patient. For example, the measurement device 110 may include a flowrate sensor for measuring the airflow of the respiration of the patient. The flowrate sensor measures, for example, a flowrate of the air passing through a tube attached to the mouse of the patient. As an example of the measurement device 110 which can measure the airflow of the respiration of the patient, there exist a device used for a positive pressure ventilation therapy, such as a continuous positive airway pressure (PAP) device and an adaptive servo ventilation (ASV) device and a device used for a polysomnography (PSG) test.

The respiratory waveform measured by the measurement device 110 may be a waveform indicating an activity (for example, a motion of the chest portion or the abdomen portion) of the patient for the respiratory effort. For example, the measurement device 110 may include a respiratory effort sensor for measuring the activity of the patient for the respiratory effort. The respiratory effort sensor measures an extension amount of a belt attached to the chest portion or the abdomen portion of the patient. As an example of the measurement device 110 which can measure the activity of the patient for the respiratory effort, there exist the device used for the PSG test and the like.

The respiratory waveform measured by the measurement device 110 may be a waveform determined on the basis of both a waveform indicating the airflow of the respiration of the patient and a waveform indicating the activity of the patient for the respiratory effort. For example, the respiratory waveform may be an average of these waveforms.

The measurement device 110 stores data indicating the measured respiratory waveform in the own memory 111. The data indicating the respiratory waveform is referred to as respiratory data 112. In a case in which the respiratory waveform is a waveform indicating the airflow of the respiration of the patient, the respiratory data 112 may indicate a measurement value of the flowrate sensor at each time point. In a case in which the respiratory waveform is a waveform indicating the activity of the patient for the respiratory effort, the respiratory data 112 may indicate the measurement value of the respiratory effort sensor at each time point. The respiratory data 112 may be the measurement value itself of the measurement device 110 or a value obtained after application of signal processing for removing noise contained in the measurement value. The respiratory data 112 may indicate a respiratory waveform estimated from a measurement value of a pulse wave sensor or the like.

The computer 100 may be a device independent of the measurement device 110. The computer 100 can use the communication device 105 to read the respiratory data 112 from the measurement device 110. The computer 100 may directly acquire the respiratory data 112 from the measurement device 110 or may acquire the respiratory data 112 via another device. For example, the computer 100 may acquire the respiratory data 112 accumulated in an external server.

The computer 100 and the measurement device 110 may be a unified device. For example, the computer 100 may be included in the CPAP device, the ASV device, or the device used for the PSG test. In other words, these devices may be configured to execute a method, which will be described later, executed by the computer 100.

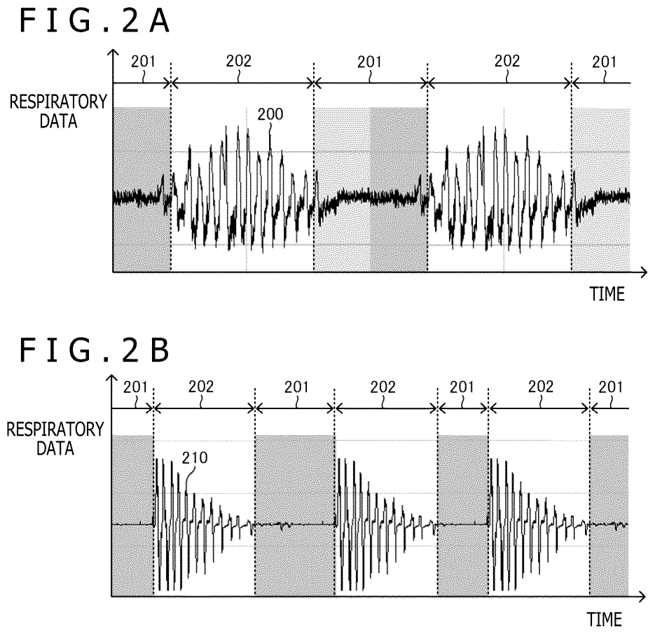

With reference to FIG. 2A and FIG. 2B, a description is now given of characteristics of the respiratory waveform of the CSR. A graph 200 of FIG. 2A indicates the respiratory data 112 of a patient contracting CSA, the respiratory data 112 being obtained while the patient is sleeping. The CSA is an example of a disease in which the CSR occurs during sleep. A graph 210 of FIG. 2B indicates the respiratory data 112 of a patient contracting OSA, the respiratory data 112 being obtained while the patient is sleeping. In each of the graphs 200 and 210, the horizontal axis indicates the time, and the vertical axis indicates the value of the respiratory data 112.

As indicated in the graphs 200 and 210, in the respiratory data 112 of the patients contracting the sleep apnea syndrome, there alternately and continuously occur a period in which the apnea or the hypopnea is present and a period in which the apnea or the hypopnea is eliminated. The period in which the apnea or the hypopnea is present is referred to as an Apnea/Hypopnea (AH) period 201 and the period in which the apnea or the hypopnea is eliminated is referred to as a respiratory period 202.

In the case of the CSR, the AH period 201 and the respiratory period 202 periodically occur. The respiratory period 202 in the case of the CSR is also referred to as a hyperventilation period. In the case of the CSR, after the respiration resumes from the apnea or the hypopnea, a respiratory amount gradually increases. After that, the respiratory amount gradually decreases, and the apnea or the hypopnea occurs again. Meanwhile, the respiratory tract is sometimes obstructed while the patient contracting the OSA is sleeping. The patient makes the respiratory effort in the state in which the respiratory tract is obstructed, thereby bringing the thoracic cavity of the patient into an extremely negative pressure state. As a result of dissolution from this negative pressure state, the respiration resumes from the apnea or the hypopnea. Thus, in the respiration in the case of the OSA, the air is suctioned into the thoracic cavity in the negative pressure state at the time of the respiration resumption, the respiratory amount hence sharply increases, and the respiratory amount returns to a normal (that is, usual) respiratory amount. After that, as the respiratory tract is gradually obstructed, the respiratory amount gradually decreases, and the apnea or the hypopnea occurs again.

Moreover, in the case of the CSR, compared with the respiration of the OSA, the length of the respiratory period 202 for one time tends to be long. Moreover, in the case of the CSR, the respiratory amplitude immediately after the respiration resumption is small while, in the respiration of the OSA, the respiratory amplitude immediately after the respiration resumption is large. Moreover, in the case of the CSR, an occurrence period (for example, a time length from the end of the respiratory period 202 to the end of the next respiratory period 202) of the AH period 201 and the respiratory period 202 tends to be long compared with that of the respiration of the OSA.

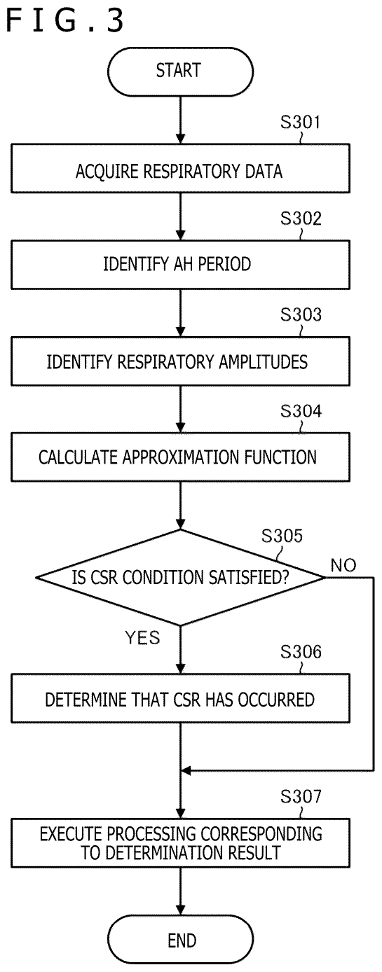

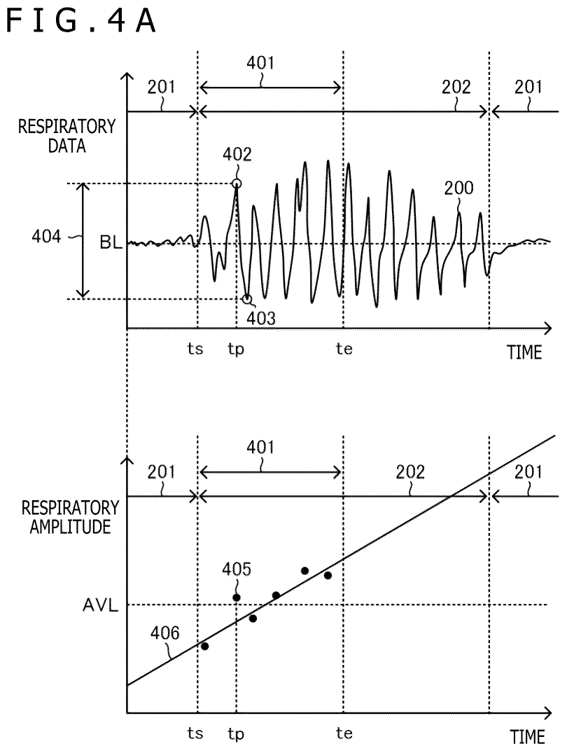

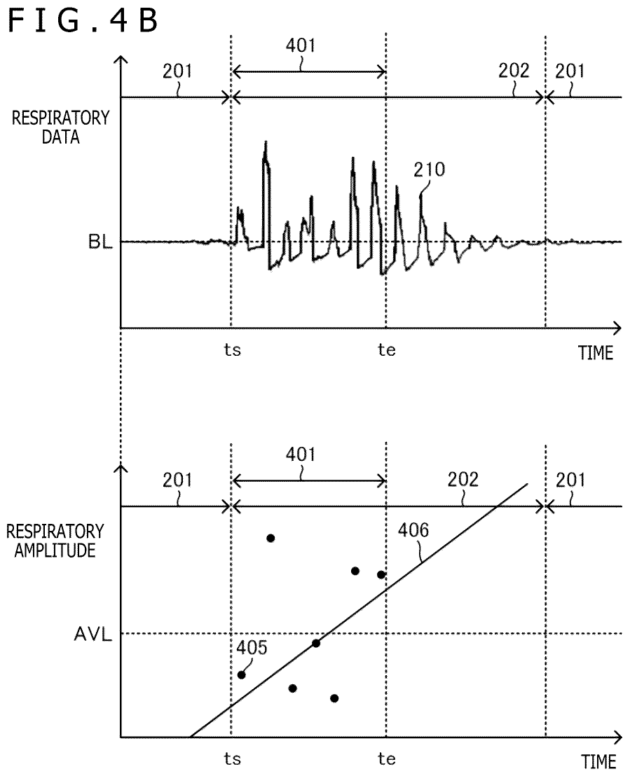

With reference to FIG. 3 to FIG. 4B, a description is now given of an example of a method for detecting the CSR. FIG. 3 illustrates a flowchart of the method for detecting the CSR. FIG. 4A and FIG. 4B illustrate a specific example in which the method of FIG. 3 is applied. Specifically, FIG. 4A illustrates an example in which the method of FIG. 3 is applied to the respiratory data 112 indicating the occurrence of the CSR, and FIG. 4B illustrates an example in which the method of FIG. 3 is applied to the respiratory data 112 indicating the occurrence of the OSA.

Each step of the method of FIG. 3 may be executed by the processor 101 executing the program read into the memory 102. Alternatively, some or all of the steps of the method of FIG. 3 may be executed by a dedicated integrated circuit such as an application-specific integrated circuit (ASIC).

The method of FIG. 3 may be executed after of the sleep of the patient ends or may be executed during the sleep of the patient. The method of FIG. 3 may be started, for example, in response to reception of an instruction from the user of the computer 100. The user of the computer 100 may be the patient himself or herself subject to the detection of the CSR. Alternatively, the user of the computer 100 may be a person (for example, a doctor) other than the patient. For example, the doctor may cause the computer 100 to execute the method of FIG. 3 for a reference of a treatment plan for the patient. Alternatively, the method of FIG. 3 may automatically be started in response to the start of the sleep of the patient.

In S301, the computer 100 acquires the respiratory data 112 indicating the respiratory waveform of the patient during sleep. For example, there is acquired the respiratory data 112 illustrated as a graph 200 on the upper side of FIG. 4A or a graph 210 on the upper side of FIG. 4B. Descriptions of the graphs 200 and 210 are similar to the descriptions of those in FIG. 2A and FIG. 2B.

In S302, the computer 100 identifies the AH period 201 which intermittently occurs in the respiratory data 112. The identification of the AH period 201 may be executed through any method and may be executed through, for example, an existing method. A period between two of the AH periods 201 adjacent to each other is identified as the respiratory period 202. Specifically, there is identified, as the respiratory period 202, a period from the end of one AH period 201 (that is, the elimination of the apnea or the hypopnea) to the start of a next AH period 201 (that is, the next occurrence of the apnea or the hypopnea). In a case in which the AH period 201 is not identified, the computer 100 determines that the apnea or the hypopnea has not occurred during sleep of the patient and may end the method of FIG. 3.

The computer 100 executes S303 and S304 for each of one or more respiratory periods 202 identified in S302.

In S303, the computer 100 identifies respiratory amplitudes 404 of the plurality of times of the respiration of the patient conducted in the one respiratory period 202. For example, as illustrated in FIG. 4A, the computer 100 identifies, as a part of one respiratory period 202, a first half 401 as a target period and identifies a maximal value 402 and a minimal value 403 of the respiratory data 112 for each of a plurality of times of the respiration included in the target period (first half 401). The maximal value 402 and the minimal value 403 identified in S303 are the maximal value 402 and the minimal value 403 of the respiratory data 112 for one time of the respiration. The maximal value 402 and the minimal value 403 are adjacent to each other. In other words, between the maximal value 402 and the minimal value 403, another maximal value or minimal value is not included. The first half 401 is a period on an earlier side of two periods obtained by dividing the one respiratory period 202 into two parts. For example, the one respiratory period 202 may be divided into two equal parts. After that, the computer 100 identifies, as the respiratory amplitude 404 of each time of the respiration, a difference between the maximal value 402 and the minimal value 403 (that is, a peak-to-peak value). Identification of a period of each time of the respiration included in the target period (first half 401) may be executed through any method and may be executed through, for example, an existing method. The maximal value 402 is the maximum value of the respiratory data 112 in the period of the one time of the respiration. The minimal value 403 is the minimum value of the respiratory data 112 in the period of the one time of the respiration.

In the examples of FIG. 4A and FIG. 4B, the peak-to-peak value (the difference between the maximal value 402 and the minimal value 403) of the respiratory data 112 is used as the respiratory amplitude 404. Alternatively, the inspiration amplitude (a difference between the maximal value 402 and a baseline value BL) of the respiratory data 112 may be used as the respiratory amplitude 404, or the expiration amplitude (a difference between the minimal value 403 and the baseline value BL) of the respiratory data 112 may be used as the respiratory amplitude 404.

The computer 100 may identify the respiratory amplitudes of the whole of the respiration conducted in the target period (first half 401). Alternatively, the computer 100 may identify the respiratory amplitudes of a part (for example, every other time or every third time) of the respiration conducted in the target period (first half 401).

In S304, the computer 100 calculates an approximation function which approximates the respiratory amplitudes of the plurality of times of the respiration identified in S303. Specifically, as illustrated in a lower side of FIG. 4A and a lower side of FIG. 4B, the computer 100 plots points 405 indicating the respiratory amplitudes 404 of the plurality of times of the respiration in a coordinate system in which a horizontal axis indicates the time and a vertical axis indicates the respiratory amplitude 404. In FIG. 4A, a reference sign is added to only one point, but six points 405 are plotted. In FIG. 4B, a reference sign is added to only one point, but seven points 405 are plotted.

A time at which the point 405 is plotted may be any time point at which each time of the respiration is conducted and may be, for example, a start time point of the respiration, an end time point of the respiration, a time point at which the maximal value 402 is taken, a time point at which the minimal value 403 is taken, and a time point at which the respiratory waveform crosses the baseline. In the example of FIG. 4A, to a time point tp at which the maximal value 402 is taken, the point 405 indicating the respiratory amplitude 404 corresponding to this time of the respiration is plotted.

In the examples of FIG. 4A and FIG. 4B, the approximation function is a first-order function, and hence, a graph 406 indicating the approximation function is a straight line. Alternatively, the approximation function may be any function such as a second-order function or a higher-order function, a trigonometric function, and an exponential function.

The computer 100 may use, for example, the least square method to calculate the approximation function. Further, the computer 100 may calculate the approximation function in such a manner as to reduce influence of outliers of the respiratory amplitudes of the plurality of times of the respiration. For example, the computer 100 may use a robust least square method to calculate the approximation function.

A description is now given of a method of calculating the approximation function through the robust least square method. First, the computer 100 uses the least square method to calculate a preliminary first-order function for approximating the respiratory amplitudes 404 of the plurality of times of the respiration. After that, the computer 100 uses the Biweight method to allocate a weight to a residual of the respiratory amplitude 404 of each time of the respiration. The residual of the respiratory amplitude 404 of the one time of the respiration is a difference between the respiratory amplitude 404 of the one time of the respiration and the value of the preliminary first-order function at a time point at which this one time of the respiration is conducted. Specifically, the computer 100 calculates a weight w(d) for a residual “d” as given by the following expression. In this expression, W is a threshold value for a permissible residual and is set in advance.

$$w ( d ) = \{ \begin{matrix}{\{ 1 - {( \frac{d}{W} )}^{2} \}}^{2} & ( \vert d \vert \leq W ) \\ 0 & ( \vert d \vert > W )\end{matrix} \qquad [ Math . 1 ]$$

After that, the computer 100 calculates a first-order function which minimizes a square sum of values each obtained by multiplying the residual of the respiratory amplitude 404 of each time of the respiration by the weight w(d). This first-order function is the approximation function calculated in S304.

With the method described above, the robust least square method is used to reduce the influence of the outliers of the respiratory amplitudes of the plurality of times of the respiration. Alternatively, another method may be used to reduce the influence of outliers. For example, there may be calculated an approximation function which excludes the respiratory amplitudes 404 larger in residual than a threshold value (for example, twice or three times of the standard deviation of the residuals) and then approximates the remaining respiratory amplitudes 404.

By reducing the influence of the outliers of the respiratory amplitudes of the plurality of times of the respiration as described above, it is possible to precisely approximate the respiratory amplitudes of the plurality of times of the respiration. As a result, detection precision of the CSR described later increases.

In S305, the computer 100 determines whether or not a predetermined condition relating to the approximation function calculated in S304 is satisfied. In a case in which it is determined that this condition is satisfied (“YES” in S305), the computer 100 causes the processing to transition to S306 and, otherwise (“NO” in S305), the computer 100 causes the processing to transition to S307. In S306, the computer 100 determines that the CSR has occurred in the patient. Thus, the condition which is used in S305 and relates to the approximation function is referred to as a CSR condition.

The CSR condition may include such a condition that the gradient of the approximation function at a specific time point is positive. This condition is referred to as a gradient condition. In a case in which the approximation function is the first-order function, the gradient at a freely-selected time point is a constant value. As illustrated in FIG. 4A, in the case of the CSR, the respiratory amplitude 404 gradually increases in the target period (first half 401), and hence, the gradient of the graph 406 (straight line) is positive. Meanwhile, in the case of the OSA, the respiratory amplitude 404 in the target period (first half 401) sharply increases and then gradually decreases, and hence, the gradient of the graph 406 (straight line) is negative.

The CSR condition may include a condition relating to the value of the approximation function at a specific time point. This condition is referred to as an approximation value condition. For example, the approximation value condition may include such a condition that the value of the approximation function at the start time point ts of the target period (first half 401) is smaller than an average AVL of the respiratory amplitudes 404 of the plurality of times of the respiration. In place of or in addition to this, the approximation value condition may include such a condition that the value of the approximation function at an end time point te of the target period (first half 401) is larger than the average AVL of the respiratory amplitudes 404 of the plurality of times of the respiration. As illustrated in FIG. 4A and FIG. 4B, the CSR satisfies this condition, but the OSA does not satisfy this condition.

The CSR condition may include a condition based on a difference between the respiratory amplitudes 404 of the plurality of times of the respiration identified in S303 and the value of the approximation function calculated in S304. For example, the CSR condition may include such a condition that a square mean of the difference between the respiratory amplitude 404 of at least one time of the respiration of the plurality of times of the respiration identified in S303 and the value of the approximation function is smaller than a threshold value. This condition is referred to as a residual condition. As described above, the residual is the difference between the respiratory amplitude 404 and the value of the approximation function.

In the residual condition, the square mean of the residuals may be calculated after there are excluded outliers of the residuals of the respiratory amplitudes 404 of the plurality of times of the respiration identified in S303. For example, there may be excluded, as the outlier, the maximum value of the residuals of the respiratory amplitudes 404 of the plurality of times of the respiration. Alternatively, in a case in which the residuals of the respiratory amplitudes 404 of the plurality of times of the respiration are larger than the threshold value (for example, twice or three times of the standard deviation of the residuals), these residuals may be excluded as the outliers.

A description is now given of a determination method for the threshold value compared with the a square mean of the residuals in the residual condition. First, the computer 100 calculates an average of “n” (for example, n=3) of the largest respiratory amplitudes 404 in the respiratory period 202, multiplies this average by a predetermined coefficient (for example, 0.3), and sets, as the threshold value, the square of this product.

In the case of CSR, the respiratory amplitude 404 gradually increases in the target period (first half 401), and the square mean of the residuals is small. Meanwhile, in the case of the OSA, the respiratory amplitude 404 sharply increases in the target period (first half 401), and hence, the square mean of the residuals is large.

The CSR condition may include only one of the gradient condition, the approximation value condition, and the residual condition described above, only two thereof, or all of the these three conditions. In a case in which the CSR condition includes two or more of the gradient condition, the approximation value condition, and the residual condition, the CSR condition may be obtained through a logical operation on these conditions. For example, in a case in which the CSR condition includes the gradient condition and the approximation value condition, the CSR condition may be satisfied in a case in which at least one of the gradient condition and the approximation value condition is satisfied (that is, the logical OR) or may be satisfied in a case in which both the gradient condition and the approximation value condition are satisfied (that is, the logical AND). The same applies to a case in which the CSR condition includes the gradient condition and the residual condition and a case in which the CSR condition includes the approximation value condition and the residual condition. In a case in which the CSR condition includes the gradient condition, the approximation value condition, and the residual condition, the CSR condition may be satisfied in a case in which at least one of these three conditions is satisfied (that is, the logical OR) or may be satisfied in a case in which all of these three conditions are satisfied (that is, the logical AND). Further, the CSR condition may include, in addition to at least one of the gradient condition, the approximation value condition, and the residual condition described above, a condition which is not described above. For example, the CSR condition may include a condition relating to the length of the respiratory period 202 or may include such a condition that the various conditions described above are continuously satisfied in a plurality of the respiratory periods 202.

In S307, the computer 100 executes processing corresponding to the determination result in S305. For example, the computer 100 may output the state of the occurrence of the CSR in a case in which the CSR is determined to have occurred. For example, the computer 100 may display the occurrence of the CSR on the display device 104. On the basis of this display, the user (for example, the patient or the doctor) of the computer 100 can recognize the occurrence of the CSR. In addition to or in place of the display of the determination result, the computer 100 may store the determination result in the secondary storage device 106 or may transmit the determination result to another device.

In a case in which the method of FIG. 3 is to be executed during the sleep of the patient, the computer 100 may change the pressure (that is, therapy pressure) of the air supplied to the patient, according to the determination result. For example, the computer 100 may maintain the therapy pressure in a case in which it is determined that the CSR has occurred and may increase the therapy pressure in a case in which it is determined that the CSR has not occurred but the obstructive apnea has occurred.

In the examples of FIG. 4A and FIG. 4B, the first half 401 of the respiratory period 202 is set to the target period, and the approximation function which approximates the respiratory amplitudes 404 of the respiration conducted in the target period is used to detect the CSR. As described with reference to FIG. 4A, the characteristics of the CSR are likely to appear in the first half 401 of the respiratory period 202. Thus, the first half 401 of the respiratory period 202 is set as the target period, thereby making it possible to precisely detect the CSR.

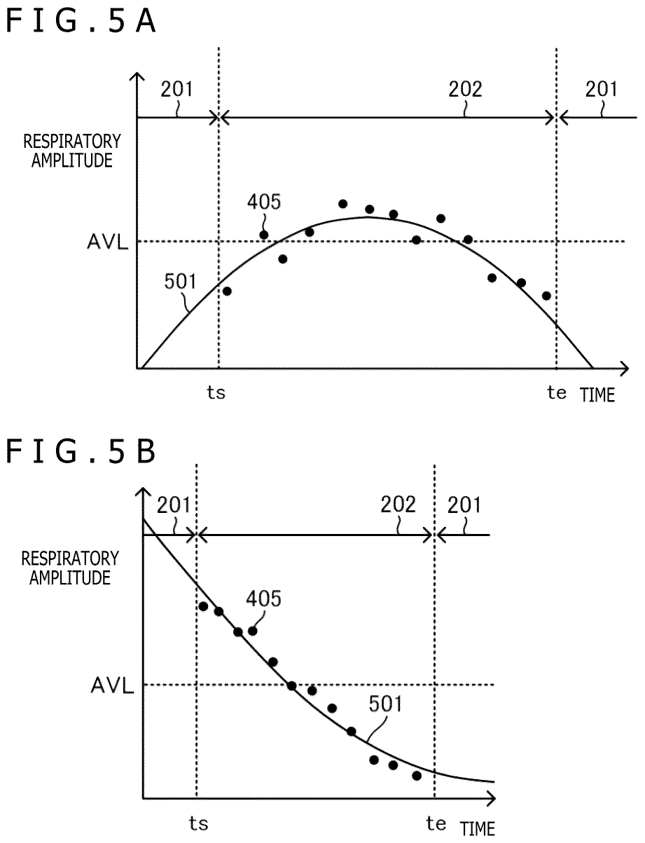

With reference to FIG. 5A and FIG. 5B, a description is given of modification examples of the specific examples of FIG. 4A and FIG. 4B. Specifically, FIG. 5A illustrates an example in which the method of FIG. 3 is applied to the respiratory data 112 indicating the occurrence of the CSR, and FIG. 5B illustrates an example in which the method of FIG. 3 is applied to the respiratory data 112 indicating the occurrence of the OSA. In FIG. 5A and FIG. 5B, the target period is the entire respiratory period 202, and the approximation function is a second-order function. A graph 501 indicating the approximation function is a parabola.

In this case, the gradient condition may include such a condition that the gradient of the approximation function at the start time point ts of the target period (respiratory period 202) is positive. The approximation value condition may include such a condition that the value of the approximation function at the start time point ts of the target period (respiratory period 202) is smaller than the average AVL of the respiratory amplitudes 404 of the plurality of times of the respiration. Further, the CSR condition may include a condition relating to the axis of the approximation function (second-order function). For example, the CSR condition may include such a condition that the axis of the approximation function is included in a range at the center of the respiratory period 202. The range of the center of the respiratory period 202 may be a range which continues in each of the forward direction and the backward direction from the time at the center of the respiratory period 202 by a predetermined length (for example, 10% or 20% of the respiratory period 202). Alternatively, the range of the center of the respiratory period 202 may be a range obtained by excluding, from the respiratory period 202, a portion having a predetermined length (for example, 10% or 20% of the respiratory period 202) from the start time of the respiratory period 202 or a range obtained by excluding, from the respiratory period 202, a portion having a predetermined length (for example, 10% or 20% of the respiratory period 202) to the end time of the respiratory period 202.

As described above, the detection of the CSR through the method of FIG. 3 is executed by analyzing the respiratory data 112 in the time domain. Thus, compared with a method of executing analysis in the frequency domain, a use amount of the memory and a calculation amount of the processor can be suppressed, thereby making it possible to efficiently detect the CSR. Further, while the method of executing the analysis in the frequency domain is influenced by a frequency peak caused by noise caused by the body movement of the patient or the like, the method of executing the analysis in the time domain is less likely to be influenced by such noise.

### Summary of Embodiments

### (Item 1)

Medical equipment for detecting abnormal respiration of a patient during sleep, including:
an identifier configured to identify respiratory amplitudes of a plurality of times of respiration conducted by the patient in a respiratory period from elimination of apnea or hypopnea to a next occurrence of the apnea or the hypopnea;
a calculator configured to calculate an approximation function that approximates the respiratory amplitudes of the plurality of times of the respiration; and
a determiner configured to determine that the abnormal respiration has occurred in the patient in a case in which a predetermined condition relating to the approximation function is satisfied.

### (Item 2)

The medical equipment according to item 1,
in which the predetermined condition includes a condition based on a difference between the respiratory amplitudes of the plurality of times of the respiration and a value of the approximation function.

### (Item 3)

The medical equipment according to item 2,
in which the predetermined condition includes such a condition that a square mean of a difference between the respiratory amplitude of at least one time of the respiration of the plurality of times of the respiration and the value of the approximation function is smaller than a threshold value.

### (Item 4)

The medical equipment according to any one of items 1 to 3,
in which the predetermined condition includes such a condition that a gradient of the approximation function at a specific time point is positive.

### (Item 5)

The medical equipment according to any one of items 1 to 4,
in which the calculator calculates the approximation function in such a manner as to reduce influence of an outlier of the respiratory amplitudes of the plurality of times of the respiration.

### (Item 6)

The medical equipment according to item 5,
in which the calculator uses a robust least square method to calculate the approximation function.

### (Item 7)

The medical equipment according to any one of items 1 to 6,
in which the plurality of times of the respiration are the respiration conducted in a part of the respiratory period.

### (Item 8)

The medical equipment according to any one of items 1 to 7,
in which the respiratory amplitudes are each a difference between a maximal value and a minimal value of respiratory data of the patient.

### (Item 9)

The medical equipment according to any one of items 1 to 8,
in which the identifier identifies the respiratory amplitudes on the basis of at least one of respiratory data indicating an airflow of the respiration of the patient or respiratory data indicating an activity of the patient for respiratory effort.

### (Item 10)

A non-transitory storage medium storing a program for causing a computer to function as each section of the medical equipment according to any one of items 1 to 9.

### (Item 11)

A method for detecting abnormal respiration of a patient during sleep, including:
identifying, by an identifier, respiratory amplitudes of a plurality of times of respiration conducted by the patient in a respiratory period from elimination of apnea or hypopnea to a next occurrence of the apnea or the hypopnea;
calculating, by a calculator, an approximation function that approximates the respiratory amplitudes of the plurality of times of the respiration; and
determining, by a determiner, that the abnormal respiration has occurred in the patient in a case in which a predetermined condition relating to the approximation function is satisfied.

The present disclosure is not limited to the embodiments described above and can be modified and changed in various ways within the gist of the disclosure.

1. Medical equipment for detecting abnormal respiration of a patient during sleep, comprising:
an identifier configured to identify respiratory amplitudes of a plurality of times of respiration conducted by the patient in a respiratory period from elimination of apnea or hypopnea to a next occurrence of the apnea or the hypopnea;
a calculator configured to calculate an approximation function that approximates the respiratory amplitudes of the plurality of times of the respiration; and
a determiner configured to determine that the abnormal respiration has occurred in the patient in a case in which a predetermined condition relating to the approximation function is satisfied.

2. The medical equipment according to claim 1,
wherein the predetermined condition includes a condition based on a difference between the respiratory amplitudes of the plurality of times of the respiration and a value of the approximation function.

3. The medical equipment according to claim 2,
wherein the predetermined condition includes such a condition that a square mean of a difference between the respiratory amplitude of at least one time of the respiration of the plurality of times of the respiration and the value of the approximation function is smaller than a threshold value.

4. The medical equipment according to claim 1,
wherein the predetermined condition includes such a condition that a gradient of the approximation function at a specific time point is positive.

5. The medical equipment according to claim 1,
wherein the calculator calculates the approximation function in such a manner as to reduce influence of an outlier of the respiratory amplitudes of the plurality of times of the respiration.

6. The medical equipment according to claim 5,
wherein the calculator uses a robust least square method to calculate the approximation function.

7. The medical equipment according to claim 1,
wherein the plurality of times of the respiration are the respiration conducted in a part of the respiratory period.

8. The medical equipment according to claim 1,
wherein the respiratory amplitudes are each a difference between a maximal value and a minimal value of respiratory data of the patient.

9. The medical equipment according to claim 1,
wherein the identifier identifies the respiratory amplitudes on a basis of at least one of respiratory data indicating an airflow of the respiration of the patient or respiratory data indicating an activity of the patient for respiratory effort.

10. A non-transitory storage medium storing a program for causing a computer to function as each section of the medical equipment according to claim 1.

11. A method for detecting abnormal respiration of a patient during sleep, comprising:
identifying, by an identifier, respiratory amplitudes of a plurality of times of respiration conducted by the patient in a respiratory period from elimination of apnea or hypopnea to a next occurrence of the apnea or the hypopnea;
calculating, by a calculator, an approximation function that approximates the respiratory amplitudes of the plurality of times of the respiration; and
determining, by a determiner, that the abnormal respiration has occurred in the patient in a case in which a predetermined condition relating to the approximation function is satisfied.
